(19) United States
(12) Patent Application Publication
(10) Pub. No.: US 2020/0090817 A1
(43) Pub. Date: Mar. 19, 2020

(54) SYSTEM AND METHOD FOR SECURE DRUG DISCOVERY INFORMATION PROCESSING

(71) Applicant: Innoplexus AG, Escborn (DE)

(72) Inventors: Gunjan Bhardwaj, Koenigstein (DE); Om Prakash, Dhanbad (IN)

(21) Appl. No.: 16/367,852

(22) Filed: Mar. 28, 2019

Related U.S. Application Data
(60) Provisional application No. 62/664,484, filed on Apr. 30, 2018.

(51) Int. Cl. G16H 70/40; H04L 9/06; G06F 16/36

(52) U.S. Cl. CPC G16H 70/40; H04L 9/0643; H04L 2209/38; G06F 16/367; H04L 9/0637

(57) ABSTRACT

A system for secure drug discovery information processing over blockchain based platform, the system including a database and a processor. The processor to receive data record from plurality of data records and metadata associated with data record from the database, wherein the data record corresponds to one of a predefined data type recognized by the blockchain based platform; retrieve ontologies from amongst plurality of ontologies, based on the metadata of the data record; measure term frequency of keywords in the retrieved ontology against term frequency of keywords in the data record; validate the data record to belong to domain of the retrieved ontology, if the keywords from the retrieved ontology are present in the data record above a predetermined value; and extract value features from the validated data record to determine an association of the data record to a node in a network map of biomedical entities.

### CROSS-REFERENCE TO RELATED APPLICATIONS

This is a non-provisional patent application based upon a provisional patent application no. U.S. 62/664,484 as filed on Apr. 30, 2018, and claims priority under 35 U.S.C. 199(e).

### TECHNICAL FIELD

The present disclosure relates generally to drug discovery information processing; and more specifically, to systems for secure drug discovery information processing over blockchain based platforms. Furthermore, the present disclosure relates to methods for secure drug discovery information processing over blockchain based platforms. Moreover, the present disclosure relates to computer readable medium containing program instructions for execution on computer systems, which when executed by a computer, cause the computer to perform aforementioned methods.

### BACKGROUND

Typically, drug discovery and development process involves screening or testing of large compound libraries, numbering millions of chemical compounds for biological activity at any one of hundreds of molecular targets in order to find potential new drugs, or lead compounds. Active compounds, or hits, from the aforesaid screening are obtained to further categorize or classify a type of finding. As a result, there is lack of advancement in drug discovery and development process.

Furthermore, drug discovery process is costly and time-consuming. One of the major limitations that researchers and scientists face during the drug discovery process is consuming a vast amount of data available in relation to specified subject matter. Moreover, researchers and/or companies tend to spend time on findings which are already existent but unknown to the researcher and/or companies. Furthermore, there is uncertainty as to whether a particular hypotheses or experimental finding is authentic.

Conventionally, medical journals and research publications have been the primary source of experimental findings and hypotheses for researchers and scientists. However, authenticating or validating the research publications suffers various drawbacks. The review of research publications is time consuming and dependent on a skillset of a reviewer.

Therefore, in the light of foregoing discussion, there exists a need to overcome the aforementioned drawbacks associated with the drug discovery and development process.

### SUMMARY

The present disclosure seeks to provide a system for secure drug discovery information processing over a blockchain based platform. The present disclosure also seeks to provide a method for secure drug discovery information processing over a blockchain based platform. The present disclosure also seeks to provide a computer readable medium containing program instructions for execution on a computer system, which when executed by a computer, cause the computer to perform aforementioned method.

The present disclosure seeks to provide a solution to the existing problem of lack of advancement in drug discovery and development process. An aim of the present disclosure is to provide a solution that overcomes at least partially the problems encountered in prior art, and provides a time efficient, resource efficient, and secure drug discovery information processing.

In one aspect, an embodiment of the present disclosure provides a system for secure drug discovery information processing over a blockchain based platform, the system comprising:

a database to store a plurality of data records and related metadata, and a plurality of ontologies; and
a processor to:
receive a data record from the plurality of data records and a metadata associated with the data record from the database, wherein the data record corresponds to one of a predefined data type recognized by the blockchain based platform;
retrieve one or more ontologies from amongst the plurality of ontologies, based on the metadata of the data record;
measure a term frequency of keywords in the retrieved ontology against a term frequency of keywords in the data record;
validate the data record to belong to a domain of the retrieved ontology, if the keywords from the retrieved ontology are present in the data record above a predetermined value; and
extract one or more value features from the validated data record to determine an association of the data record to a node in a network map of biomedical entities.

In another aspect, an embodiment of the present disclosure provides a method for secure drug discovery information processing over a blockchain based platform, the method comprising:

(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.

In yet another aspect, an embodiment of the present disclosure provides a computer readable medium containing program instructions for execution on a computer system, which when executed by a computer, cause the computer to perform a method, wherein the method is implemented via a system comprising:

(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.

Embodiments of the present disclosure substantially eliminate or at least partially address the aforementioned problems in the prior art, and enable time efficient, resource efficient, and secure drug discovery information processing over the blockchain based platform.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a block diagram of a system for secure drug discovery information processing over a blockchain based platform, in accordance with an embodiment of the present disclosure; and

FIG. 2 is an illustration of steps of a method for secure drug discovery information processing over a blockchain based platform, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a system for secure drug discovery information processing over a blockchain based platform, the system comprising:

a database to store a plurality of data records and related metadata, and a plurality of ontologies; and
a processor to:
receive a data record from the plurality of data records and a metadata associated with the data record from the database, wherein the data record corresponds to one of a predefined data type recognized by the blockchain based platform;
retrieve one or more ontologies from amongst the plurality of ontologies, based on the metadata of the data record;
measure a term frequency of keywords in the retrieved ontology against a term frequency of keywords in the data record;
validate the data record to belong to a domain of the retrieved ontology, if the keywords from the retrieved ontology are present in the data record above a predetermined value; and
extract one or more value features from the validated data record to determine an association of the data record to a node in a network map of biomedical entities.

In another aspect, an embodiment of the present disclosure provides a method for secure drug discovery information processing over a blockchain based platform, the method comprising:

(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.

In yet another aspect, an embodiment of the present disclosure provides a computer readable medium containing program instructions for execution on a computer system, which when executed by a computer, cause the computer to perform a method, wherein the method is implemented via a system comprising:

(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.

The present disclosure relates to a system and a method for secure drug discovery information processing over a blockchain based platform. Beneficially, the system accelerates drug discovery process to provide a mechanism for secure and validated data record associations. The associations within a plurality of data record allows in creation of a network map of biomedical entities. The network map of the data record is beneficially modified to include new findings and their associations within existing network map. The secure drug discovery system is further connected to a block chain platform. Beneficially, the blockchain platform is configured to store hash of the data record and other related data on a cryptographic ledger to secure the data record. Therefore, the present disclosure provides a secure drug discovery system for associating validated data records.

Beneficially, the system comprising the processor to process the drug discovery information requires RAM (Random Access Memory) with less storage space. Moreover, the system minimizes the resource consumption of the processor. Consequently, the RAM is available for performing other tasks of the processor and further increases computational speed of the processor. Additionally, the system requires less computing power compared to high computing power required by existing systems.

The present disclosure provides the system for secure drug discovery information processing over the blockchain based platform. The system is a collection of one or more interconnected programmable and/or non-programmable components configured to associate data records to network map of biomedical entities to enable secure drug discovery information processing. Examples include programmable and/or non-programmable components, such as processors, memories, connectors, cables and the like. Moreover, the programmable components are configured to store and execute one or more computer instructions.

Throughout the present disclosure, the term “drug discovery information” refers to information for gaining knowledge of or ascertaining the existence of something previously unknown or unrecognized related to a substance intended for use in the diagnosis, cure, mitigation, or prevention of a disease. Optionally, the information is indicative of a drug, a pathway, a target and a disease and is also indicative of inter-relationships therewith. In an example, a relationship between a drug and a disease could be ‘causes’, ‘inhibits’, ‘catalyzes’ and so on. The drug discovery information is processed by the system and stored over the blockchain based platform. Moreover, the processing of the drug discovery information comprises receiving the drug discovery information, measuring the frequency of keywords in the drug discovery information, validating the domain of the drug discovery information, and determining association of the drug discovery information in the network map of biomedical entities.

The term, “blockchain based platform” refers to a ledger of operations and/or contracts. In this regard, the ledger is consensually shared and synchronized across multiple sites, institutions or geographies. Pursuant to embodiments of the present disclosure, the blockchain based platform refers to a databank of entries, wherein the entries comprise the drug discovery information therein. Moreover, the blockchain based platform is consensually shared and synchronized in a decentralized form across a plurality of computing nodes. Optionally, such computing nodes are established across different locations and operated by different users. Beneficially, the blockchain based platform eliminates the need of a central authority to maintain and protect against manipulation. Specifically, the entries comprising the operation records in the blockchain based platform are monitored publicly, thereby making the blockchain based platform robust against attacks. Therefore, the drug discovery information stored over the blockchain based platform is secure.

It will be appreciated that the plurality of computing nodes in the distributed ledger may access each of the entries in the blockchain based platform and may own an identical copy of each of the entries. Notably, an alteration made to the blockchain based platform is reflected almost instantly to each of the plurality of computing nodes. Subsequently, an alteration (such as recordal of an entry in the blockchain based platform) is done when all or some of the plurality of computing nodes perform a validation with respect to the alteration. In such case, the entry is recorded (namely, added) in the blockchain based platform in an immutable form when at least a threshold number of computing nodes from the plurality of computing nodes reach a consensus that the entry is valid. Alternatively, recording of the entry is denied when the threshold number of computing nodes reach a consensus that the entry is invalid. In an example, the threshold number of computing nodes to reach a consensus may be fifty-one per cent (51%) of the plurality of computing nodes. Optionally, information in the blockchain based platform is stored securely using cryptography techniques. Beneficially, the blockchain based platform allows reliable and transparent recordal of the entries, in that the operation records (for example, exchange of a technical resource over the data communication network) are permanently recorded and may not be capable of alterations. Thus, the blockchain based platform provides greater transparency, enhanced security, improved traceability, increased efficiency and speed of operations.

The system comprises the database to store the plurality of data records and related metadata, and the plurality of ontologies. Throughout the present disclosure, the term “database” as used herein refers to an organized body of digital information regardless of the manner in which the data record, related metadata and the plurality of ontologies thereof are represented. Optionally, the database may be hardware, software, firmware and/or any combination thereof. For example, the organized body of data record, related metadata and the plurality of ontologies may be in the form of a table, a map, a grid, a packet, a datagram, a file, a document, a list or in any other form. The database includes any data storage software and systems, such as, for example, a relational database like IBM DB2 and Oracle 9. Optionally, the database may be used interchangeably herein as database management system, as is common in the art. Furthermore, the database management system refers to the software program for creating and managing one or more databases.

Moreover, the term, “plurality of data records” refers to a set of files in which information is recorded, wherein the information is recorded as a data class. Some examples of various data classes are text data, tabular data, image data, and so forth. Thus, the plurality of data records may be in any suitable file formats depending upon the data class in which the information is recorded. Moreover, the plurality of data records further comprises associated attributes that relate to visual appearance thereof. In an example, the associated attribute may include a structure relating to the plurality of data records such as a layout, a design, and so forth. In another example, the associated attributes may include a format relating to the plurality of data records such as font, color, and image, and so forth. Optionally, each of the plurality of data records adheres to a subject area and/or a domain associated therewith. More optionally, each of the plurality of data records adheres to a language such as English, German, Chinese and the like. Optionally, each of the plurality of data records may be saved as a uniquely named file in one or more databases. More optionally, each of the plurality of data records may be received from a user via a user device such as cellular phones, personal digital assistants (PDAs), handheld devices, wireless modems, laptop computers, personal computers and the like.

Furthermore, the term, “related metadata” refers to a data about one or more features and properties associated with each of the plurality of data records. Moreover, the metadata comprises a collection of words associated with the data records such as entities of the data records, concepts of the data records, categories of the data record and the like. Additionally, the metadata provides understanding about the information in the data records. In an example, metadata associated with a data record comprises a date of creation of the data record, computational size of the data record, an author of the data record, a file type of data record, a word count of the data record, a language of the data record and the like. In another example, metadata associated with a data record comprises 24 February as the date of creation, 20 kilobytes as the computational size of the data record, ‘ABC’ as the author of the data record, Microsoft Word Document as the file type, 350 words as the word count of the data record, and English as the language of the data record.

Furthermore, the term, “plurality of ontologies” refers to a set of words associated as concepts, categories, and so forth of a given domain and/or a given subject. Typically, an ontology defines properties associated with the set of words and relations therebetween in the given domain. Moreover, the plurality of ontologies has knowledge pertaining to the utilization of the set of words based on properties of the words and relations between the words, in the given domain. In other words, the plurality of ontologies has semantic relations between the set of words relating to concepts, categories, and so forth in the given domain, wherein the semantic relations define at least one of: properties, relations, and utilization associated with the set of words. Optionally, each ontology of the plurality of ontologies relates to a specific domain such that each ontology has the set of words of the specific domain. In an example, a first ontology has a set of words of life science domain, a second ontology has a set of words of computer domain, a third ontology has a set of words of bio-technology domain, a fourth ontology has a set of words of medical science domain, a fifth ontology has a set of words of finance domain.

The system comprises the processor. Throughout the present disclosure, the term “processor” refers to a computational element that is operable to respond to and processes instructions that drive the system. Optionally, the processor includes, but is not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the term “processor” may refer to one or more individual processors, processing devices and various elements associated with a processing device that may be shared by other processing devices. Additionally, the one or more individual processors, processing devices and elements are arranged in various architectures for responding to and processing the instructions that drive the system.

Moreover, the processor is operable to receive the data record from the plurality of data records and the metadata associated with the data record from the database. It is to be understood that, the processor is communicatively coupled to the database to receive the data record and the metadata associated with the data record. Optionally, the data processing arrangement is communicatively coupled to the database via one or more data communication networks. The one or more data communication networks may be a collection of individual networks, interconnected with each other and functioning as a single large network. Such individual networks may be wired, wireless, or a combination thereof. Examples of such individual networks include, but are not limited to, Local Area Networks (LANs), Wide Area Networks (WANs), Metropolitan Area Networks (MANs), Wireless LANs (WLANs), Wireless WANs (WWANs), Wireless MANs (WMANs), the Internet, second generation (2G) telecommunication networks, third generation (3G) telecommunication networks, fourth generation (4G) telecommunication networks, fifth generation (5G) telecommunication networks and Worldwide Interoperability for Microwave Access (WiMAX) networks.

Furthermore, the data record corresponds to one of the predefined data type recognized by the blockchain based platform. It is to be understood that, a specific data record stored in the database corresponds to a specific data type. However, the blockchain based platform recognizes particular data types of the data record referred to as the predefined data type. In an embodiment, the predefined data type comprises experimental reports, publications, research articles. Optionally, it is to be understood that, the experimental reports, the publications, the research articles do not limit the scope of predefined data type. Optionally, the metadata of the data record may be input by a user. More optionally, the metadata of the data record may be extracted from the data record by the processor.

Furthermore, the processor is operable to retrieve one or more ontologies from amongst the plurality of ontologies, based on the metadata of the data record. As previously mentioned, the metadata comprises the collection of words associated with the data records and the plurality of ontologies comprises the set of words of a given domain and/or a given subject. In a case, the collection of words associated with metadata of a data record are used by the processor to retrieve one or more ontologies having exact words and/or similar words as the collection of words associated with the data records. In an example, similar words refer to synonyms of the collection of words. Typically, the collection of words associated with the data records is mapped with the words in the one or more ontologies to identify and retrieve the one or more ontologies. In an example, the metadata of a data record comprises a collection of words such as cancer, lung cancer and the like. In such a case, the one or more ontologies having words such as cancer, lung cancer, tumor, neoplasm adenocarcinoma are retrieved by the processor. As previously mentioned, metadata comprise one or more features and properties are associated with each of the plurality of data records. In a case, one or more features and properties of the data records are used by the processor to retrieve the one or more ontologies having similar features and properties. In an example, a data record has author associated therewith. In such a case, the processor retrieves one or more ontologies having a set of words associated with the same author as that of the data record. Optionally, the aforementioned one or more data communication networks enable the processor to retrieve the one or more ontologies from the database.

Moreover, the processor is operable to measure the term frequency of keywords in the retrieved ontology against the term frequency of keywords in the data record. It is to be understood that, the set of words associated as concepts, categories, and so forth of domains and/or subjects are referred to here as the keywords in the plurality of ontologies. It is to be understood that, the collection of words associated with the data records in the metadata are referred to here as the keywords in the data record. The term, “term frequency” refers to reoccurrence of a keyword in an environment comprising the keywords, the environment herein being the retrieved ontology and the data record. Typically, each keyword of the keywords in the data record is mapped with each word of the set of words in the retrieved ontology. Moreover, each word of the set of words in the retrieved ontology which are mapped to the keywords in the data record is thereby referred as the keyword in the retrieved ontology. Typically, keywords in the data record may exist at multiple locations. The keywords at each location of the multiple locations are mapped with each word of the set of words in the retrieved ontology. Therefore, words in the set of words may be mapped multiple times to be referred to as the keywords. The mapping of keywords multiple times indicates the term frequency of the keywords in the retrieved ontology. Optionally, the term frequency of the keywords in the retrieved ontology and the term frequency of the keywords in the data record may be a mathematical number such that a mathematical number represents the number of times a specific keyword in retrieved ontology is mapped by the keywords in the data record.

Furthermore, the processor is operable to validate the data record to belong to the domain of the retrieved ontology, if the keywords from the retrieved ontology are present in the data record above the predetermined value. As mentioned previously, each ontology of the plurality of ontologies is associated to a domain and/or a subject. The processor retrieves one or more ontologies from amongst the plurality of ontologies based on the metadata of the data record. However, each of the one or more retrieved ontologies may be associated with different domains and/or subjects. Therefore, the processor validates the data record to identify the domain associated with the data record. It is to be understood that, the keywords from the retrieved ontology which are mapped multiple times by the keywords in the data record have a value associated, wherein the value is based on the number of times the keywords are mapped. If the value associated with the keywords from the retrieved ontology is above the predetermined value the data record is validated to belong to the domain of the keywords in the retrieved ontology.

In an embodiment, the processor is configured to validate the data record by calculating a confidence score based on a presence of keywords in the data record, from the retrieved ontology. The term, “confidence score” relates to a grade, points, a percentage or any other way of scoring the keyword based on the presence of keywords in the data record. Moreover, it is to be understood that, if the confidence score is above a predefined score the data record is validated to belong to the domain of the keywords in the retrieved ontology. In an example, a keyword in the data record is present 10 times and the predefined score is 8. In such a case, the confidence score is said to be 10 which is greater than the predefined score. Therefore, in such a case, the data record is validated to belong to the domain of the keywords in the retrieved ontology.

Moreover, the processor is operable to extract one or more value features from the validated data record to determine the association of the data record to the node in the network map of biomedical entities. Throughout the present disclosure, the term “network map” refers to one or more connections between biomedical entities such that each connection between two biomedical entities represents a relationship between the two biomedical entities. It is to be understood that, the network map comprises one or more nodes such that each node represents a biomedical entity. Optionally, the network map of biomedical entities displays various stages in a drug discovery process and their results. More optionally, the nodes in the network map relates to the different stages in drug discovery process. In an example, a first node may represent a disease and the other nodes connected to the first node may represent the cause of the disease, effect of the disease, symptoms of the disease and the like.

Optionally, the network map may have a tree structure, wherein the node includes a pointer (namely, address) to a parent node. It will be appreciated that the node may or may not have a child node. Consequently, the node may or may not include a pointer to the child node. Moreover, the node may have 0, 1, 2, 3, and so on, number of child node associated therewith. Typically, the tree structure is instigated by a root node (namely, the starting point of the tree), wherein the root node is the highest-level node. The tree structure is terminated by leaf nodes (namely, the ending point of the tree), wherein the leaf nodes are the bottom-level nodes.

The association of the data record to the node represents a relation between the data record and the biomedical entities existing in the network map as one or more nodes. In an example, the data record comprises information about lung cancer and causes of lung cancer. In such a case, the data record is associated to a node representing the causes of lung cancer. The one or more value features of the validated data record enables in determining an association of the data record with the nodes in the network map of biomedical entities. Optionally, the said association may be visualized on a graphical user interface.

In an embodiment, the one or more value features extracted from the validated data record is one of: genetic association score, somatic association score, specifically targeted experimental records mapping to a predetermined entity. The term “genetic association score” refers to observations of a change in genetic variants associated with a disease or trait. The term “somatic association score” refers to an account of probability of occurrence of somatic mutations (mutations in sexual hormones, ovule, sperms and so forth) in a body. Furthermore, somatic mutations are changes to genetics of a body of a multicellular organism which are not passed on to offspring through germlines. In addition, mutations may include transformations, structural changes, behavioral changes and so forth. The specifically targeted experimental records may be medical observations, reading of medical experiments and the like which are mapped to the predetermined entity.

In an embodiment, the processor is configured to validate the data record by determining a pre-existing association for the extracted one or more value features of the data record. The pre-existing association may refer to relationships existing between the extracted one or more value features of the data record. The relationships may be predefined by the processor to be related to a particular domain which enables the data record to be validated to belong to the domain of the retrieved ontology.

In an embodiment, the processor is further configured to create the network map of biomedical entities using associations within the plurality of data records. Optionally, associations within the plurality of data records may be used by the processor to create a new network map of biomedical entities. Optionally, associations within the plurality of data records may be used by the processor to add the data record to already existing network map of entities.

In an embodiment, the processor is further configured to: generate a hash of the data record, the metadata and the determined association of the data record to the node in the network map; and store the hash on a cryptographic ledger associated with the blockchain platform along with a timestamp. Optionally, hash refers to a unique identification value which uniquely represents a specific data record, metadata associated with the specific data record, and the determined association of the specific data record. It is to be understood that, the hash is different for every set of data record, metadata and the determined association of the data record. Optionally, the processor involves the use of hash generation algorithms such as SHA1, SHA2 for generating hash. Typically, hash is of a definite length. The cryptographic ledger is to be referred to as a blockchain. It is to be understood that, the hash generated by the processor is stored on the blockchain as a block along with the timestamp. Moreover, based on any changes in the data records, and/or metadata, and/or determined association of the data record a subsequent block is created. In an example, an owner of the data record is changed. In such a case, a subsequent block is created which represents change in the data record.

In an embodiment, the cryptographic ledger is a distributed ledger. The generation of hash and storing of hash on the distributed ledger makes the data record and the determined association immutable. Moreover, the data record cannot be manipulated any further since the hash along with the timestamp shall always be present in form of the block in the distributed ledger.

Moreover, the present description also relates to the method as described above. The various embodiments and variants disclosed above apply mutatis mutandis to the method.

Optionally, the predefined data type comprises: experimental reports, publications, research articles.

Optionally, the one or more value features extracted from the validated data record is one of: genetic association score, somatic association score, specifically targeted experimental records mapping to a predetermined entity.

Optionally, the method further comprises:

generating a hash of the data record, the metadata and the determined association of the data record to the node in the network map; and
storing the hash on a cryptographic ledger along with a timestamp.

Optionally, the cryptographic ledger is a distributed ledger.

Optionally, validating the data record comprises:

calculating a confidence score based on a presence of keywords in the data record, from the retrieved ontology; and/or
determining a pre-existing association for the extracted one or more value features of the data record.

Optionally, the method further comprises creating the network map of biomedical entities using associations within the plurality of data records.

### DETAILED DESCRIPTION OF THE DRAWINGS

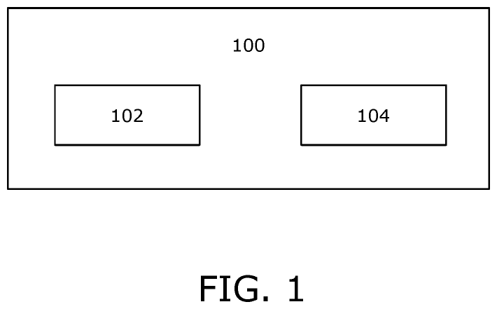

Referring to FIG. 1, there is shown a block diagram of a system 100 for secure drug discovery information processing over a blockchain based platform, in accordance with an embodiment of the present disclosure. As shown, the system 100 comprises a database 102 to store a plurality of data records and related metadata, and a plurality of ontologies; and a processor 104.

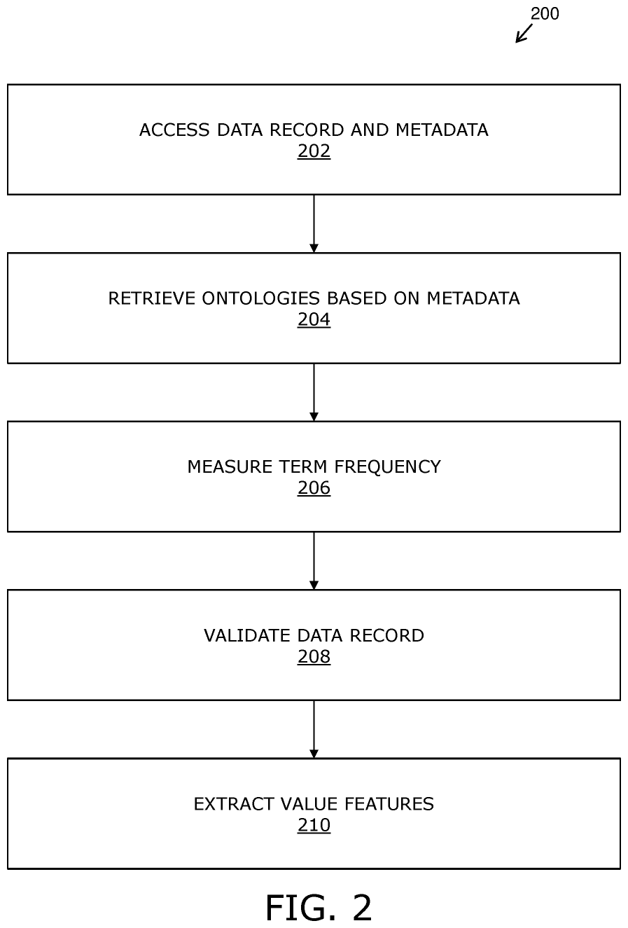

Referring to FIG. 2, there is shown an illustration of steps of a method 200 for secure drug discovery information processing over a blockchain based platform, in accordance with an embodiment of the present disclosure. At a step 202, a data record and a metadata associated with the data record is accessed, wherein the data record corresponds to one of a predefined data type recognized by the blockchain based platform. At a step 204, one or more ontologies from amongst a plurality of ontologies are retrieved, based on the metadata of the data record. At a step 206, a term frequency of keywords in the retrieved ontology is measured against a term frequency of keywords in the data record. At a step 208, the data record to belong to a domain of the retrieved ontology is validated, if the keywords from the retrieved ontology are present in the data record above a predetermined value. At a step 210, one or more value features are extracted from the validated data record to determine an association of the data record to a node in a network map of biomedical entities.

The steps 202 to 210 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system for secure drug discovery information processing over a blockchain based platform, the system comprising:
a database to store a plurality of data records and related metadata, and a plurality of ontologies; and
a processor to:
receive a data record from the plurality of data records and a metadata associated with the data record from the database, wherein the data record corresponds to one of a predefined data type recognized by the blockchain based platform;
retrieve one or more ontologies from amongst the plurality of ontologies, based on the metadata of the data record;
measure a term frequency of keywords in the retrieved ontology against a term frequency of keywords in the data record;
validate the data record to belong to a domain of the retrieved ontology, if the keywords from the retrieved ontology are present in the data record above a predetermined value; and
extract one or more value features from the validated data record to determine an association of the data record to a node in a network map of biomedical entities.

2. A system according to claim 1, wherein the predefined data type comprises: experimental reports, publications, research articles.

3. A system according to claim 1, wherein the one or more value features extracted from the validated data record is one of: genetic association score, somatic association score, specifically targeted experimental records mapping to a predetermined entity.

4. A system according to claim 1, wherein the processor is further configured to:
generate a hash of the data record, the metadata and the determined association of the data record to the node in the network map; and
store the hash on a cryptographic ledger associated with the blockchain platform along with a timestamp.

5. A system according to claim 4, wherein the cryptographic ledger is a distributed ledger.

6. A system according to claim 1, wherein the processor is configured to validate the data record by:
calculating a confidence score based on a presence of keywords in the data record, from the retrieved ontology; and/or
determining a pre-existing association for the extracted one or more value features of the data record.

7. A system according to claim 1, wherein the processor is further configured to create the network map of biomedical entities using associations within the plurality of data records.

8. A method for secure drug discovery information processing over a blockchain based platform, the method comprising:
(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.

9. A method according to claim 8, wherein the predefined data type comprises: experimental reports, publications, research articles.

10. A method according to claim 8, wherein the one or more value features extracted from the validated data record is one of: genetic association score, somatic association score, specifically targeted experimental records mapping to a predetermined entity.

11. A method according to claim 8, further comprising:
generating a hash of the data record, the metadata and the determined association of the data record to the node in the network map; and
storing the hash on a cryptographic ledger along with a timestamp.

12. A method according to claim 11, wherein the cryptographic ledger is a distributed ledger.

13. A method according to claim 8, wherein validating the data record comprises:
calculating a confidence score based on a presence of keywords in the data record, from the retrieved ontology; and/or
determining a pre-existing association for the extracted one or more value features of the data record.

14. A method according to claim 8, further comprising creating the network map of biomedical entities using associations within the plurality of data records.

15. A computer readable medium containing program instructions for execution on a computer system, which when executed by a computer, cause the computer to perform a method, wherein the method is implemented via a system comprising:
(i) accessing a data record and a metadata associated with the data record, wherein the data record corresponds to one of a predefined data type recognized by the blockchain based platform;
(ii) retrieving one or more ontologies from amongst a plurality of ontologies, based on the metadata of the data record;
(iii) measuring a term frequency of keywords in the retrieved ontology against a term frequency of keywords in the data record;
(iv) validating the data record to belong to a domain of the retrieved ontology, if the keywords from the retrieved ontology are present in the data record above a predetermined value; and
(v) extracting one or more value features from the validated data record to determine an association of the data record to a node in a network map of biomedical entities.
